(19) United States
(12) Patent Application Publication
(10) Pub. No.: US 2008/0031944 A1
(43) Pub. Date: Feb. 7, 2008

(54) Stabilization of lorazepam

(75) Inventors: Larry Bereuter, Columbia Heights, MN (US); David K. Brown, Plymouth, MN (US); Derek Moe, Maple Grove, MN (US)

Correspondence Address: CEPHALON, INC., 41 MOORES ROAD, PO BOX 4011, FRAZER, PA, 19355, US

(73) Assignee: CIMA LABS INC., Eden Prairie, MN (US)

(21) Appl. No.: 11/888,788

(22) Filed: Aug. 2, 2007

Related U.S. Application Data
(60) Provisional application No. 60/835,744, filed on Aug. 4, 2006.

(51) Int. Cl. A61K 9/20; A61K 31/5513; A61K 9/14

(52) U.S. Cl. 424/464; 424/493; 424/494; 424/499; 514/221

(57) ABSTRACT

This invention relates to orally disintegrable, lorazepam-containing dosage forms which are storage stable and disintegrable within about 90 seconds or less. In one embodiment, there is provided a storage stable, orally disintegrable dosage form comprising: protected lorazepam particles comprising lorazepam and polymeric material having a glass transition temperature of about 65° C. or above. Also disclosed is a method of producing a storage stable lorazepam containing tablet.

### BACKGROUND OF THE INVENTION

Sometimes for formulation chemists, as for all of us, it just doesn't pay to get out of bed in the morning. Lorazepam, 7-chloro-5-(2-chlorophenyl)-1,3-dihydro-3-hydroxy-2H-1,4-benzodiazepin-2-one[846-49-1], is known to be used to treat anxiety and to prevent convulsions. It is available in many different delivery formats including oral concentrates, injectables, oral solutions, and oral swallowable tablets. The latter are currently approved in 0.5, 1, and 2 milligram strengths.

Since this particular active pharmaceutical ingredient (“API”) had already been produced in such a wide variety of delivery formats, and as various formats for orally disintegrable tablets (“ODT”) (used interchangeably with orally disintegrating tablets) were known, it was reasonable to assume that one could produce an orally disintegrable dosage form capable of delivering lorazepam. As with many such assumptions, however, this turned out to be incorrect. It was discovered that lorazepam is unstable with such typically used ODT excipients as mannitol and super disintegrants such as crosslinked PVP (crosslinked polyvinylpyrolidone a/k/a crospovidone or PVPP). In addition, flavors used in orally disintegrable tablets, something not normally necessary in tablets to be swallowed, also could destabilize lorazepam.

As a result, attempting to formulate orally disintegrable dosage forms including lorazepam routinely resulted in a reduction of potency of 16% or greater (much of the time 20% or greater) when measured during a forced degradation study. To make matters worse, various techniques for isolating lorazepam from potentially destabilizing excipients did not help. For example, one possible coating which may be useful in some orally disintegrating tablets is an acrylic based material sold under the trade name EUDRAGIT® E-100. This material is particularly useful in taste masking in that it dissolves at a pH generally below about 6.5, i.e., once the coated material enters the stomach. This type of coating would be desirable for lorazepam, even though lorazepam is not particularly bad tasting as it is meant to be freely available once it reaches the stomach. However, when lorazepam was coated with EUDRAGIT® E-100, it was unstable as well. Moreover, lorazepam is not very stable when exposed to water and might have stability problems with other conventional solvents. This suggested that coating and granulation techniques often used in the pharmaceutical industry would only further complicate the problem. One knowing this would limit themselves to either completely dry processes or the use of more exotic solvents, both of which could impart their own unique problems.

Thus, there remains a need for orally disintegrating tablets containing lorazepam.

### SUMMARY OF THE INVENTION

In one embodiment, there is provided a storage stable, orally disintegrable dosage form comprising: protected lorazepam particles comprising lorazepam and polymeric material having a glass transition temperature of about 65° C. or above. Such polymeric materials can include, without limitation, a cellulose based material, povidone (“PVP”) or a poloxamer (synthetic copolymers of ethylene oxide and propylene oxide, many sold under the trademark PLURONIC®). The protected lorazepam particles are present in an amount sufficient to provide a therapeutically effective amount of lorazepam ranging from about 0.1 to about 100 mg per dosage form. The dosage form also comprises at least one disintegrant which is crosslinked PVP, croscaramellose salt such as croscaramellose sodium, or a starch glycolate such as sodium starch glycolate, and/or an effervescent couple. In a preferred embodiment, the dosage form further comprises at least one carbohydrate based filler. The dosage form is capable of disintegrating in the mouth of a patient, within about 90 seconds or less as measured by the procedures set forth in the U.S.P. 29, chapter <701> (2006) entitled “Disintegration” for uncoated tablets (referred to herein as the “U.S.P.”) and has a loss of potency of about 15% or less as determined by forced degradation.

In one embodiment, the protected lorazepam particles are produced by layering an API-containing layer onto a support optionally followed by coating. In another embodiment, the protected lorazepam particles are produced by granulation, optionally followed by coating.

In still another embodiment, the present invention provides a storage stable, orally disintegrable tablet (“ODT”) comprising protected lorazepam particles comprising lorazepam and a polymeric material having a glass transition temperature of about 65° C. or more. In a preferred embodiment, these polymers are a cellulose based material or PVP. The protected lorazepam particles are present in an amount sufficient to provide a therapeutically effective amount of lorazepam ranging from about 0.1 to about 100 mg per tablet. At least one disintegrant is provided and is selected from the group consisting of crosslinked PVP, croscarmellose salt, a starch glycolate and/or an effervescent couple. The tablet also comprises at least one carbohydrate based filler. The tablet has a loss of potency of about 10.5% or less as determined by forced degradation and is either bioequivalent to nonorally disintegrable tablets containing lorazepam as described herein and/or is capable of disintegrating in within about 60 seconds or less as determined by the U.S.P.

Also contemplated are methods of making these dosage forms which include the steps of producing protected lorazepam particles either by granulation or by a layering, either followed optionally by a coating process, mixing the protected lorazepam particulates with at least one carbohydrate based filler, at least one disintegrant selected from crosslinked PVP, a croscaramellose salt or a starch glycolate and/or an effervescent couple, and optionally other excipients which, when compressed, produce an orally disintegrable dosage form capable of disintegrating in about 90 seconds or less as measured by U.S.P., more preferably in about 60 seconds or less, and is storage stable as measured by forced degradation, and compressing same to form a tablet. Methods of administering these dosage forms to a patient in need thereof are also contemplated.

In a particularly preferred embodiment, the process of granulation and/or layering is accomplished using water. Despite the use of this solvent/carrier, the resulting tablets have excellent storage stability and disintegration times.

### DETAILED DESCRIPTION OF THE INVENTION

While the specification concludes with the claims particularly pointing and distinctly claiming the invention, it is believed that the present invention will be better understood from the following description. All percentages and ratios used herein are by weight of the total dosage form, or coated lorazepam particle, as the context requires, unless otherwise designated. All measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

“Storage stable” in accordance with the present invention means having a loss of potency of lorazepam of about 15% or less when subjected to a forced degradation study. A forced degradation study in accordance with the present invention is accomplished on tablets by placing unpackaged tablets in an open flask (five 2-mg tablets or eight 1-mg tablets) followed by placing the flask in a convection oven at 80 degrees Celsius for five days. Humidity is not controlled beyond the normal laboratory environmental control. Protected lorazepam particles, both those made by granulation and those made by layering, may also be subjected to the same conditions, in amounts of 100 mg to 270 mg depending on dose. Percent loss of lorazepam potency is measured by comparison of the HPLC assay for the concentration of lorazepam in the dosage forms or protected particles before and after forced degradation. Ultimately, the comparison is between the amount, by weight of lorazepam remaining after forced degradation. Any other analytical technique which can provide this information may also be used.

In a more preferred embodiment, the dosage forms in accordance with the present invention have a loss of potency of about 13% or less and even more preferably about 10.5% or less. In one preferred embodiment, the storage stable dosage forms of the present invention have a loss on potency similar to or better than that which was obtained by doing a comparable test on an equal strength of lorazepam swallowable tablets (swallowable without disintegration or dissolution) sold under the trade name ATIVAN®, which, as tested, had a maximum loss of potency of 10.4% for 1 milligram tablets and 3.6% for 2 milligram tablets.

In contrast to the dosage forms of the present invention, when testing ODT tablets made by the inventors from lorazepam with various excipients and production techniques in various formats, it was found that the loss of potency generally was about 15% or greater. Indeed, the majority of such formulations provided a loss of potency of about 20% or greater and the lowest loss of potency was 16.7%. See Tables I-IV below.

TABLE I
Prototype Formulas and Forced Degradation Results

| | 1820-13 | 1820-14 | 1820-15 | 1820-16 | 1820-17 | 1820-18 |
|---|---|---|---|---|---|---|
| Lorazepam, USP | 0.50 | 0.50 | 0.50 | 0.50 | 0.50 | 0.50 |
| Granular Lorazepam, polacrilin | | | | | | |
| Granular Lorazepam, sod.bicarb | | | | | | |
| Mannitol EZ | 44.25 | 41.25 | | | | |
| Fast-Flo Lactose (316) | | | | | | |
| Emdex Dextrates | | | | | | |
| Powdered Mannitol | 25.00 | 25.00 | 69.25 | 66.25 | 71.25 | 64.25 |
| Microcrystalline Cellulose | 15.00 | 15.00 | 15.00 | 15.00 | 15.00 | 15.00 |
| Croscaramellose Sodium | 10.00 | 10.00 | 10.00 | 10.00 | 10.00 | 10.00 |
| Sodium Starch Glycolate | | | | | | |
| Polacrilin Potassium | 2.00 | 5.00 | 2.00 | 5.00 | | 5.00 |
| Polacrilin Potassium, Dried | | | | | | |
| Polacrilin Potassium, Ground | | | | | | |
| Sodium Bicarbonate | | | | | | |
| Orange Flavor | 0.75 | 0.75 | 0.75 | 0.75 | 0.75 | 0.75 |
| Sucralose | 0.50 | 0.50 | 0.50 | 0.50 | 0.50 | |
| Aspartame | | | | | | 2.50 |
| Magnesium Stearate | 2.00 | 2.00 | 2.00 | 2.00 | 2.00 | 2.00 |
| Silicon Dioxide | | | | | | |
| | 100.00 | 100.00 | 100.00 | 100.00 | 100.00 | 100.00 |
| Forced Degradation Result: | −33.4% | −34.6% | −21.0% | −19.5% | −37.1% | −20.6% |
| 2 mg Ativan Result: | −3.6% | | | | | |
| *2 mg Mylan Result: | −14.7% | | | | | |

| | 1820-19 | 1820-20 | 1820-21 | 1820-24 |
|---|---|---|---|---|
| Lorazepam, USP | 0.50 | 0.50 | 0.50 | 0.50 |
| Granular Lorazepam, polacrilin | | | | |
| Granular Lorazepam, sod.bicarb | | | | |
| Mannitol EZ | | | | |
| Fast-Flo Lactose (316) | | | | |
| Emdex Dextrates | | | | |
| Powdered Mannitol | 66.25 | 64.25 | 66.25 | 64.25 |
| Microcrystalline Cellulose | 15.00 | 15.00 | 15.00 | 15.00 |
| Croscaramellose Sodium | 10.00 | 10.00 | 10.00 | |
| Sodium Starch Glycolate | | | | 10.00 |
| Polacrilin Potassium | 5.00 | | | |
| Polacrilin Potassium, Dried | | 5.00 | 5.00 | 5.00 |
| Polacrilin Potassium, Ground | | | | |
| Sodium Bicarbonate | | | | |
| Orange Flavor | 0.75 | 0.75 | 0.75 | 0.75 |
| Sucralose | 0.50 | | 0.50 | |
| Aspartame | | 2.50 | | 2.50 |
| Magnesium Stearate | 2.00 | 2.00 | 2.00 | 2.00 |
| Silicon Dioxide | | | | |
| | 100.00 | 100.00 | 100.00 | 100.00 |
| Forced Degradation Result: | −24.8% | −23.4% | −21.4% | −21.1% |

*“MYLAN ™” refers to generic equivalent to ATIVAN ® produced by Mylan Pharmaceuticals, Inc.

TABLE II
Prototype Formulas and Forced Degradation Results

| | 1820-24 | 1820-26 | 1820-27 | 1820-28 |
|---|---|---|---|---|
| Lorazepam, USP | 0.50 | 0.50 | 0.50 | 0.50 |
| Granular Lorazepam, polacrilin | | | | |
| Granular Lorazepam, sod.bicarb | | | | |
| Mannitol EZ | | | | |
| Fast-Flo Lactose (316) | | | | |
| Emdex Dextrates | | | | |
| Powdered Mannitol | 64.25 | 69.25 | 69.25 | 66.25 |
| Microcrystalline Cellulose | 15.00 | 15.00 | 15.00 | 15.00 |
| Croscaramellose Sodium | | | | |
| Sodium Starch Glycolate | 10.00 | 10.00 | 10.00 | 10.00 |
| Polacrilin Potassium | | 2.00 | | |
| Polacrilin Potassium, Dried | 5.00 | | | |
| Polacrilin Potassium, Ground | | | 2.00 | |
| Sodium Bicarbonate | | | 0.00 | 5.00 |
| Orange Flavor | 0.75 | 0.75 | 0.75 | 0.75 |
| Sucralose | 0.00 | 0.50 | 0.50 | 0.50 |
| Aspartame | 2.50 | | | |
| Magnesium Stearate | 2.00 | 2.00 | 2.00 | 2.00 |
| Silicon Dioxide | | | | |
| | 100.00 | 100.00 | 100.00 | 100.00 |
| Forced Degradation Result: | −21.5% | −21.6% | −22.2% | −20.7% |
| 2 mg Ativan Result: | −1.9% | | | |
| 2 mg Mylan Result: | −14.3% | | | |
| 1 mg Ativan Result: | −9.40% | | | |
| | 1820-29 | 1820-30-1 | 1820-31 | 1820-32 |
| Lorazepam, USP | 0.50 | 0.50 | 0.50 | 0.50 |
| Granular Lorazepam, polacrilin | | | | |
| Granular Lorazepam, sod.bicarb | | | | |
| Mannitol EZ | | | | |
| Fast-Flo Lactose (316) | 44.25 | 45.00 | | |
| Emdex Dextrates | | | 44.25 | |
| Powdered Mannitol | 25.00 | 25.00 | 25.00 | 68.95 |
| Microcrystalline Cellulose | 15.00 | 15.00 | 15.00 | 15.00 |
| Croscaramellose Sodium | | | | |
| Sodium Starch Glycolate | 10.00 | 10.00 | 10.00 | 10.00 |
| Polacrilin Potassium | 2.00 | 2.00 | 2.00 | 5.00 |
| Polacrilin Potassium, Dried | | | | |
| Polacrilin Potassium, Ground | | | | |
| Sodium Bicarbonate | | | | |
| Orange Flavor | 0.75 | | 0.75 | 0.75 |
| Sucralose | 0.50 | 0.50 | 0.50 | 0.50 |
| Aspartame | | | | |
| Magnesium Stearate | 2.00 | 2.00 | 2.00 | 2.00 |
| Silicon Dioxide | | | | 0.30 |
| | 100.00 | 100.00 | 100.00 | 103.00 |
| Forced Degradation Result: | −22.2% | −20.0% | −19.3% | −22.2% |

TABLE III
Prototype Formulas and Forced Degradation Results

| | 1820-41 | 1820-42 | 1820-44 | 1820-45 |
|---|---|---|---|---|
| Lorazepam, USP | 0.5 | 1 | | |
| Granular Lorazepam, polacrilin | | | 10 | 10 |
| Granular Lorazepam, sod.bicarb | | | | |
| Mannitol EZ | | | | 31.45 |
| Fast-Flo Lactose (316) | | | | |
| Emdex Dextrates | | | | |
| Powdered Mannitol | 58.95 | 63.45 | 56.45 | 25 |
| Microcrystalline Cellulose | 20 | 20 | 20 | 15 |
| Croscaramellose Sodium | | | | |
| Sodium Starch Glycolate | 10 | 10 | 10 | 10 |
| Polacrilin Potassium | | | | |
| Polacrilin Potassium, Dried | | | | |
| Polacrilin Potassium, Ground | 2 | 2 | | |
| Sodium Bicarbonate | 5 | | | 5 |
| Orange Flavor | 0.75 | 0.75 | 0.75 | 0.75 |
| Sucralose | 0.5 | 0.5 | 0.5 | 0.5 |
| Aspartame | | | | |
| Magnesium Stearate | 2 | 2 | 2 | 2 |
| Silicon Dioxide | 0.3 | 0.3 | 0.3 | 0.3 |
| | 100.00 | 100.00 | 100.00 | 100.00 |
| Forced Degradation Result: | −28.7% | −21.1% | −23.0% | −23.6% |
| 2 mg Ativan Result: | −2.0% | | | |
| 2 mg Mylan Result: | −15.2% | | | |
| 1 mg Ativan Result: | −10.4% | | | |
| | 1820-46 | 1820-48 | 1820-49 | 1820-50 |
| Lorazepam, USP | | | | |
| Granular Lorazepam, polacrilin | 20 | | | |
| Granular Lorazepam, sod.bicarb | | 10 | 10 | 20 |
| Mannitol EZ | 31.45 | | 31.45 | 31.45 |
| Fast-Flo Lactose (316) | | | | |
| Emdex Dextrates | | | | |
| Powdered Mannitol | 25 | 56.45 | 25 | 25 |
| Microcrystalline Cellulose | 10 | 20 | 15 | 10 |
| Croscaramellose Sodium | | | | |
| Sodium Starch Glycolate | 5 | 10 | 10 | 5 |
| Polacrilin Potassium | | | | |
| Polacrilin Potassium, Dried | | | | |
| Polacrilin Potassium, Ground | | | | |
| Sodium Bicarbonate | 5 | | 5 | 5 |
| Orange Flavor | 0.75 | 0.75 | 0.75 | 0.75 |
| Sucralose | 0.5 | 0.5 | 0.5 | 0.5 |
| Aspartame | | | | |
| Magnesium Stearate | 2 | 2 | 2 | 2 |
| Silicon Dioxide | 0.3 | 0.3 | 0.3 | 0.3 |
| | 100.00 | 100.00 | 100.00 | 100.00 |
| Forced Degradation Result: | −26.0% | −21.9% | −22.5% | −16.7% |

TABLE IV
Prototype Formulas and Forced Degradation Results

| | 1608-50 | 1820-04 | 1820-05 | 1820-06 | 1820-07 |
|---|---|---|---|---|---|
| Lorazepam, DSP | 0.50 | 0.50 | 0.50 | 0.50 | 0.50 |
| Granular Lorazepam, | | | | | |
| polacrilin | | | | | |
| Granular Lorazepam, | | | | | |
| sod.bicarb | | | | | |
| Mannitol EZ | 44.75 | 46.25 | 46.25 | 46.25 | 46.25 |
| Fast-Flo Lactose (316) | | | | | |
| Emdex Dextrates | | | | | |
| Powdered Mannitol | 25.00 | 25.00 | 25.00 | 25.00 | 25.00 |
| Microcrystalline | 15.00 | 15.00 | 15.00 | 15.00 | 15.00 |
| Cellulose | | | | | |
| Croscaramellose | | 10.00 | 10.00 | | |
| Sodium | | | | | |
| Sodium Starch | | | | 10.00 | 10.00 |
| Glycolate | | | | | |
| Polacrilin Potassium | | | | | |
| Polacrilin Potassium, | | | | | |
| Dried | | | | | |
| Polacrilin Potassium, | | | | | |
| Ground | | | | | |
| Sodium Bicarbonate | | | | | |
| Orange Flavor | 0.75 | 0.75 | 0.75 | 0.75 | 0.75 |
| Sucralose | | 0.50 | 0.50 | 0.50 | 0.50 |
| Aspartame | 2.00 | | | | |
| Magnesium Stearate | 2.00 | 2.00 | 2.00 | 2.00 | 2.00 |
| Silicon Dioxide | | | | | |
| Crospovidone | 10.00 | | | | |
| | 100.00 | 100.00 | 100.00 | 100.00 | 100.00 |
| Forced Degradation | −33.6% | −61.00% | −62.2% | −55.9% | −61.7% |
| Result: | | | | | |
| 2 mg Ativan Result: | −7.0% | | | | |

The dosage forms of the present invention thus provide greater stability than that which were achieved using many other possible ODT delivery formats attempted by the inventors.

In addition to storage stability, the dosage forms in accordance with the present invention are orally disintegrable. “Orally disintegrable” and like terms such as “orally disintegrating” in the context of the present invention means a dosage form that is disintegrable/dissolvable when placed in the mouth of a patient. This means that at least a portion of the dosage form may disintegrate and/or dissolve when, for example, placed on the tongue in a patient's mouth. The term does not include dosage forms which are designed to facilitate transfer of lorazepam across an oral mucosa such as sublingual or buccal tablets. When disintegration/dissolution of the dosage forms of the invention is achieved sufficiently, the resulting dispersion, suspension or solution of the coated lorazepam particles are then swallowed.

The dosage forms in accordance with the present invention are typically capable of disintegrating/dissolving in the mouth of a patient within about 90 seconds or less, more preferably within about 60 seconds or less, and even more preferably within about 30 seconds or less. Again, this can be measured by the procedure set forth in the U.S.P. as noted earlier. It is understood, however, that in the mouth, not all of the dissolvable material contained within the dosage form has in fact dissolved or that the dosage form has completely disintegrated. However, if the in vitro test is satisfied, dissolution and disintegration should have occurred sufficiently to allow swallowing of the resulting solution, suspension or dispersion in an organoleptically pleasant manner in the time recited.

In certain embodiments of the present invention, the storage stable orally disintegrable dosage forms of the invention are “bioequivalent” to a nonorally disintegrable dosage form containing the same dose of lorazepam. By this it is understood that conventional tests for bioequivalency reveal that the dosage form is bioequivalent, within the meaning of Title 21 and 21 C.F.R. which were in force on the date this document was first filed in a patent office. That is to say, they are within 80-125% in terms of Cmax and/or area under the curve (AUC) when compared to the comparable dose of ATIVAN swallow tablets as measured by standard protocols for bioequivalence necessary to support such a claim to the U.S. Food and Drug Administration when filing an Abbreviated New Drug Application pursuant to 21 U.S.C. § 355(j). Generally the U.S. U.S. Food and Drug Administration considers two products to be bioequivalent if the 90% confidence interval of the relative means Cₘₐₓ, AUC₍₀₋ₜ₎ and AUC₍₀₋₀₀₎ of the test (e.g. generic formulation) to reference (e.g. innovator brand formulation).

The orally disintegrable dosage forms of the present invention include protected lorazepam particles. Protected lorazepam particles comprise lorazepam and polymer having a glass transition temperature of about 65° C. or above (a “GTT65 polymer”), cellulose based materials, PVP and poloxamers that have a suitable glass transition temperature may be used. Cellulose based materials and PVP are preferred. The lorazepam and polymer may be used together in a layer, may be used together in a binder or may be separate, as in separate layers or particles and a binder. The protected lorazepam particles may also optionally include one or more coatings. The protected lorazepam particles in accordance with the present invention can be made by any number of techniques. Two such techniques, however, are layering/coating and granulation. In one embodiment protected lorazepam particles are produced by layering, often by spray coating, a solution, suspension or dispersion of lorazepam mixed with a GTT65 polymer and a solvent, (preferably an aqueous solvent), onto the surface of carrier particles, often a sugar or cellulose based sphere. This could also be done by two successive layers, one of lorazepam and one of the GTT65 polymer. Optionally, after being layered with the lorazepam containing layer, the resulting particle can be coated, or more specifically overcoated, with one or more additional coatings. The coatings may also be composed of a GTT65 polymer, preferably a cellulose based material or PVP, but that need not be the case.

A solid support or carrier particle in accordance with the present invention can be composed of any material useful for layering in accordance with this and other conventional pharmaceutical applications. These can include, without limitation, particles, crystals, granulates, capsules, mini-tablets microparticles, microgranules, microcrystals or microcapsules. Particles, granules and crystals have their traditional meaning. “Capsule” in accordance with the present invention includes generally hollow, spherical vessels such as liposomes, micelles and the like. “Micro” in the context of carrier particles means a carrier particle having a particle size of below about 50 microns. Preferably the carrier particles are substantially spherical although the particle dimensions can vary and can be, without limitation, elliptical, generally egg-shaped, rod-shaped, regular and/or irregularly shaped.

Carrier particles can be composed of any number of materials or mixtures thereof including particles created from one or more of the taste masking materials, polymers, solid dicalcium phosphate and the like. It should be noted however that a solid support is optional. Particles of lorazepam could themselves be coated with the GTT65 polymer such as a cellulose based material or PVP to produce the protected lorazepam particles.

However, in a preferred embodiment, the carrier particles are made of a sugar. “Sugar” in accordance with the present invention generally includes other forms of carbohydrate such as, for example, sugars, sugar alcohols, ketoses, saccharides, polysaccharides, oligosaccharides and the like, as well as celluloses and modified celluloses. These include, without limitation, sucrose, mannitol (spray dried and granular) lactose, and microcrystalline cellulose. Most preferred in accordance with the present invention are sucrose and microcrystalline cellulose. Useful sucrose spheres are available from Paulaur corporation, 105 Melrich Road, Cranbury, N.J. 08512. Useful microcrystalline spheres are sold by Asahi Kasei Chemicals Corp, with the following address: Hibiya-Mitsui Building 1-2 Yurakucho 1-chome, Chiyoda-ku, Tokyo 100-8440 Japan under the designation CELPHERES®.

The size of the carrier particles can vary considerably with, amongst other things, the application, volume of the carrier particles that will be used in the formulation, the type of dosage form in which it will be included, and the thicknesses of the layers that will coat it. Carrier particles that are too small can be difficult to layer. Carrier particles that are too large can be difficult to work with, can affect content uniformity and can provide an unpleasant organoleptic sensation in the mouth.

In accordance with the present invention, the carrier particle size is preferably between about 10 microns and about 1,000 microns, more preferably between about 20 microns and 600 microns. This means that at least about 90% of the carrier particles, by weight, fall within these ranges based on sieving. One preferred solid support used is sugar spheres (60/80) NF which has the following particle size distribution: 50-mesh, ≧100% through; 60-mesh, ≧90% through; 80-mesh, ≦10% through. The specification for a second preferred solid support is sugar spheres (45/60) which is 40-mesh, ≧100% through; 45-mesh, ≧90% through; 60-mesh, ≦10% through.

The lorazepam containing layer is layered over at least a portion of the carrier particle or solid support. By “layered over at least a portion” in context of any coating or layer described herein, it is understood that the complete surface area of each carrier particle or coated particle need not be covered. Indeed, while the efficiency of the system is improved considerably by the use of a substantially complete and uniform coating, it is not required that, for example, the lorazepam coating cover even a majority of the carrier particles or a majority of the surface area of the carrier particles. Preferably, however, the lorazepam containing layer covers substantially all of the carrier particles to which it is applied (it is possible to mix some coated and uncoated solid support if desired) and each successive layer preferably does the same.

It will also be appreciated that the amount of lorazepam in a layer, the amount of a GTT65 polymer in the layer and the amount of layering material used in producing the protected lorazepam particles can vary greatly. The single most important factor to be considered is the amount of lorazepam that is to be delivered in each dosage form, an amount which generally ranges from between about 0.01 to about 500 milligrams, more preferably from between about 0.10 to about 100 milligrams, and most preferably from between about 0.25 to about 10 milligrams per dosage form. The size of the dosage form and the dose may further dictate the relative concentration of lorazepam in the layering material and all of the foregoing may affect the amount of lorazepam containing layering material to be used. Generally, however, the concentration of the lorazepam in the layering mixture will range from between about 0.1 to about 20, more preferably between about 1 to about 10 and most preferably between about 3 to about 4% by weight. The amount of cellulose based material or PVP in the coating material will generally range from between about 0.3 to about 10, more preferably between about 2 to about 8 and most preferably between about 4 to about 6% by weight. The balance will be solvent and any coating excipients or other coating materials used as discussed herein. Obviously, this is not the amount of material that will be found in the dried layer as there will be little or no residual solvent.

The amount of lorazepam containing layer applied in terms of percent weight gain will generally range from between about 5 to about 100%, more preferably between about 10 to about 30% and most preferably between about 15 to about 22% by weight based on weight gain as measured against the solid support or carrier particle which is uncoated (or coated with any desirable sublayer). This is based on the dry weight of the particle and/or coating as appropriate. Note in this document that the amount of lorazepam described refers to calculations based on the weight of the free base form of the drug unless otherwise specified. The corresponding amount of salt, solvate, hydrate or other derivative may also be used as long as it provides an equivalent amount of free base as recited herein.

Any material can be used in the layering of lorazepam as long as its use is consistent with the objectives of the present invention. Thus, material which would be insoluble in the stomach, react negatively with lorazepam, such as EUDRAGIT® E-100 or which produces poor processability would be undesirable. Acrylic based materials are therefore generally less desirable. Particularly preferred layering materials in which the lorazepam can be dissolved, suspended or dispersed are GTT65 polymers. These include, without limitation, cellulose based materials and polyvinylpyrolidone (“PVP”). Particularly preferred cellulose based materials include hydroxypropylmethylcellulose (HPMC a.k.a. hypromellose), hydroxypropylcellulose (HPC), and ethylcellulose (EC) and poloxamers. In one embodiment, for example, an aqueous solution containing 5% HPMC by weight and 3.6% lorazepam by weight can be prepared and sprayed onto the surface of sugar spheres. When applied to sugar spheres (60/80) as described previously, the result was a potency of approximately 3.3%. This is the amount of drug as a percent, by weight, of the layered product. Potency may also be determined by assay.

Once layered with the lorazepam containing layer the carrier particles can optionally be further coated or over coated. Any material can be used for the coating which is consistent with the objectives of the present invention. Particularly preferred materials are the cellulose based materials and PVP described above. However, this need not be the case. For example, a layer of EUDRAGIT® E100 could be used as a coating to provide taste-masking. Alternatively, the layered particle could be coated first with a thin layer of a cellulose based material or PVP and thereafter coated with EUDRAGIT® E100, the thin coating of the cellulose based material or PVP acting as a spacing layer.

The amount of coating to be applied to the lorazepam layered carrier particles can range from 0 to 100% weight gain (which, as in all instances of weight gain, may be calculated based on the weight of the layered particles and the amount of coating material to be applied, or by weighing before and after coating). However, in a preferred embodiment, the amount of this over coating (including all coating layers if more than one) ranges from 0 to 30% weight gain and more preferably from about 5 to about 15% weight gain. This is based on the dried coated particle before and after coating.

In one particularly preferred embodiment in accordance with the present invention, the coating will comprise the same material used to dissolve, disperse or suspend the lorazepam in the lorazepam containing layer. This may provide great flexibility in terms of processing in that the coating apparatus may not need to be emptied and cleaned in between layering and subsequent coating operations. For example, the lorazepam coated sugar spheres described above can be over coated with a second coating of a 5% aqueous HMPC solution in an amount, and for a time sufficient, to achieve an overall 8.4% weight gain. This means that the weight of the sugar sphere coated with the lorazepam coating material was increased in weight by 8.4%.

The lorazepam containing layer, and indeed any over coating layer(s) as well, can be applied by any conventional coating process such as use of a fluidized bed with Wurster column where the coating material enters from the bottom of the reactor. The lorazepam is preferably dissolved, suspended or dispersed in a solvent and the resulting solution, dispersion or suspension is then coated onto the surface of the carrier particles preferably in a way which provides a substantially homogeneous coating. The solvent should be acceptable to the U.S. Food and Drug Administration or comparable government agencies and is sufficiently volatile to be removed quickly either by air drying or by use of other drying equipment at a temperature which is insufficient to cause damage to the lorazepam. The concentration of the lorazepam in the solvent will vary with the solvent, the coating material used, and whether or not a solution, suspension or dispersion is to be produced. It is understood, however, that as little solvent as is necessary should be used. Solvents in accordance with the present invention include, for example, water, alcohol, dehydrated ethanol, methanol, isopropyl alcohol, acetone, dioxane and chloroform. Water and aqueous solvents are preferred, despite the fact that lorazepam is thought to be relatively unstable when exposed to water.

In an alternate embodiment, the protected lorazepam particles of the present invention can be produced by granulation and more preferably by wet granulation. Wet granulation using water or an aqueous solvent is particularly preferred, despite the fact that lorazepam is thought to be relatively unstable when exposed to water. It is the granulation process which results in the creation of a “coating-like” structure which protects the lorazepam from the other excipients. In one embodiment, lorazepam can be dissolved, dispersed or suspended in an appropriate carrier or solvent and sprayed onto some sort of solid support in a granulator. Alternatively, the lorazepam can be placed in a granulator, wetted with a solvent and/or binder, and granulated directly. In yet another embodiment, the lorazepam can be mixed with a cogranulate material and the resulting mixture of particles cogranulated with a granulation liquid and/or granulation binder. The cogranulate approach is preferred.

The solvent used in granulation will depend on the coating/binding material to be used and the other factors described herein including granulate size, the cogranulate material, the granulation technique being used, the percentage of lorazepam to be included within the granulate and the like. In general, however, the solvents used and the GTT65 polymers as described previously in connection with layering are used to produce the granulate (in this case used as a binder).

Granulation under high shear conditions is most preferred.

Cogranulates in accordance with the present invention include, without limitation, carbonates, bicarbonates, microcrystalline cellulose, mannitol, lactose and such other carbohydrates and cellulosic materials or inert materials that do not themselves degrade lorazepam.

The granulate may be used directly out of the granulator (following drying) or may be milled and sized to obtain a desired average particle size or particle size distribution. The size range of uncoated granules may range from about 10 microns to 1000 microns. The size range of uncoated granules will more generally range from between about 44 to about 590 microns, more preferably between about 74 to about 420 microns and most preferably between about 149 to about 250 microns. The size of the granulate may also vary depending on whether or not it will be subsequently coated.

The relative proportion of lorazepam and cogranulate can vary widely. They can depend on the degree of protection that is required, the amount of lorazepam to be delivered, the size of the dosage form, the type of cogranulate used, the type of binder used the concentration of binder in the wet granulate solution and in the resulting granulate and the like. However, generally, the amount of lorazepam used is less than the amount of cogranulate. More preferably, the amount of cogranulate used in the granulate will range from between about 50 to about 99.5% by weight, more preferably between about 60 to about 90% by weight, and most preferably between about 75 to about 85% by weight based on the weight of the finished granulate. The amount of lorazepam will generally range from between about 0.5 to about 50% by weight, more preferably between about 3 to about 30% by weight, and most preferably between about 5 to about 15% by weight. The remainder will be the binder, any additional excipients and/or residual solvents.

The granulate may include additional binders or excipients found in granulates or other dosage forms such as, for example, sodium bicarbonate and polacrillin potassium. The amount of binder in the solvent mixture used applied to granulation can vary with the various conditions, equipment and materials being used.

An aqueous solution of one or more GTT65 polymer as binders in an amount of 1 to 35%, more preferably 1 to 20%, and even more preferably 1 to 15% is useful. An aqueous solution of 25% povidone by weight was found to be useful. The upper limit is dependent on the viscosity of the resulting solution and may be somewhere between 30% and 35% for aqueous povidone. The lower limit is dependent on the minimum amount of binder deemed sufficient for the purpose and on the amount of solvent needed for successful “wet massing” of the granulation. 25% has a manageable viscosity and allows the addition of sufficient binder with an appropriate amount of solvent.

For a cellulose based material used in a binder, the concentration would be less in an aqueous solution, due to viscosity constraints. A 1 to 25% solution by weight, more preferably a 1 to 15% solution by weight, and even more preferably a 1 to 10% solution by weight are useful. 5% would be near optimal for aqueous HPMC.

These granulated particles can be, as was the case of the layered particles described previously, coated or over coated with one or more additional layers. Particularly preferred coating materials are GTT65 polymers including, without limitation, cellulose based material and polyvinylpyrolidone (“PVP”). Particularly preferred cellulose based materials include hydroxypropylmethylcellulose (HPMC a.k.a. hypromellose), hydroxypropylcellulose (HPC), and ethylcellulose (EC).

However, as before, this subsequent coating layer used to over coat the granulate need not be limited to the use of cellulose based materials and/or PVP For example, a coating layer of EUDRAGIT® E100 could be used as a coating to provide taste-masking. Alternatively, the granulate could be coated first with a thin layer of a GTT65 polymer such as a cellulose based material or PVP and thereafter coated with EUDRAGIT® E100, the thin coating of the GTT65 polymer acting as a spacing layer.

The amount of coating to be applied to the lorazepam containing granulate can range from 0 to 100% weight gain (which, as in all instances of weight gain, may be calculated based on the weight of the granulate and the amount of coating material to be applied, or by weighing before and after coating). However, in a preferred embodiment, the amount of this over coating (including all coating layers if more than one) ranges form 0 to 30% weight gain and more preferably from about 5 to about 15% weight gain. This is based on the dried coated granulate before and after coating.

Thus, a protected lorazepam particle in accordance with the present invention can be, inter alia, a carrier particle layered with a layer of a GTT65 polymer mixed with lorazepam, a carrier particle layered with a GTT65 polymer mixed with lorazepam subsequently coated with one or more additional layers, a lorazepam containing granulate wherein the granulate includes, even if only as a binder, a GTT65 polymer, and such a granulate coated with one or more additional layers.

The protected lorazepam particles are generally dried before use although drying can be a passive process. Drying can be accomplished using the fluidized bed, for example, without the addition of additional solvent or coating material. These may be stored in open air, may be exposed to an oven or placed in a generally warm environment. Care should be taken, however, to ensure that the degree of heating is not sufficient to cause premature or excessive degradation of the lorazepam.

The amount of protected lorazepam particulate used in the dosage forms in accordance with the present invention can vary with a number of factors, not the least of which is the sound medical judgment of the treating physician, the intended dose, and the number of dosage forms which are to be administered per dose and per day and the size of each dosage form. Preferably, each dose of lorazepam is divided into no more than two dosage forms and most preferably is administered in a single dosage form. The amount of protected lorazepam particles in accordance with the present invention will also vary with the amount of lorazepam in each particle, the size of the particles, the type of other excipients to be used, the thickness of the coating and the like. Generally, however, the amount of protected lorazepam particles per dosage form ranges from between about 4% by weight to about 40% by weight. More preferably, the amount of protected lorazepam particles per dosage form ranges from between about 10% by weight to about 20% by weight.

The balance of the dosage forms in accordance with the present invention comprise conventional excipients used in the industry and in particular in the ODT industries. Particularly advantageous in accordance with the present invention is the use of materials which generally could not have been used in combination with swallowable tablets of lorazepam. These otherwise contraindicated excipients include mannitol, super disintegrants and flavoring agents.

Super disintegrants useful in accordance with the present invention include those known as super disintegrants. These include, without limitation, crosslinked PVP, croscaramellose salts such as croscaramellose sodium, starch glycolates such as sodium starch glycolate.

Where such super disintegrants are used, they are traditionally found in an amount of between about 1 and about 15%, more preferably between about 4 and about 8%, and most preferably between about 5 and about 7% by weight of the finished dosage form. In addition to, instead of any portion of, or instead of any super disintegrant, the dosage forms in accordance with the present invention may include at least one effervescent couple.

Effervescent couples are made from a reaction of a soluble acid source and a metal carbonate or bicarbonate. The acid sources or acid may be any which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations of the present invention were intended to be dissolved in a glass of water. Acid anhydrides and acid salts of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate and amorphous calcium carbonate. These effervescent couples may be provided in an amount of between about 3% and about 50% by weight of the dosage form, more preferably between about 3% and about 25% by weight.

A dosage form according to the present invention may also include suitable noneffervescent, nonsuper disintegrants. Nonlimiting examples of such noneffervescent disintegration agents include: microcrystalline cellulose, starches, corn starch, potato starch and modified starches thereof, clays, such as bentonite, alginates, gums such as agar, guar, locust bean, karaya, pecitin and tragacanth. These disintegrants may comprise up to about 20 weight percent and preferably between about 2% and about 10% of the total weight of the dosage form.

Carbohydrate based fillers which may be used in accordance with the present invention include sugars, sugar alcohols, ketoses, celluloses, starches, and the like. These can include but are not limited to spray dried lactose monohydrate, anhydrous fast flow lactose, sucrose, dextrose, mannitol, spray dried mannitol, sorbitol, starch, cellulose such as microcrystalline cellulose and maltodextrins. These are collectively referred to as carbohydrates herein.

The carbohydrates used may be nondirect compression and/or direct compression carbohydrates and mixtures thereof. Nondirect compression carbohydrates generally, at least when formulated, have flow and/or compression characteristics which make them impractical for use in the high speed tableting processes without augmentation or adjustment. For example, a formulation may not flow sufficiently well and therefore a glidant such as for example silicon dioxide may be added. Direct compression carbohydrates, by contrast, do not require similar allowances. They generally have compressibility and flow characteristics which allow them to be used directly.

It is noted that, depending upon the method by which formulations are made, nondirect compression carbohydrates may be imparted with properties of direct compression carbohydrates. The reverse is also true. As a general matter, nondirect compression carbohydrates tend to have a relatively smaller particle size when compared to direct compression carbohydrates. However, certain carbohydrates, such as spray dried mannitol, have relatively smaller particle size and yet are often directly compressible, depending on how they are further processed. There are also relatively large nondirect compression carbohydrates known as well. The amount of carbohydrates used in accordance with the present invention range from between about 40 to about 90%, more preferably from about 60 to about 80% and most preferably from about 65 to about 75%. In one embodiment, the majority of the carbohydrate used is a nondirect compression carbohydrate. In an alternate embodiment, the minority of the carbohydrate used is a nondirect compression carbohydrate. Indeed in one preferred embodiment, the amount of nondirect compression carbohydrate ranges from between about 15 to about 35% by weight.

Diluents and fillers which may be used in accordance with the present invention include for example dihydrated or anhydrous dibasic calcium phosphate, tricalcium phosphate, calcium carbonate, and calcium sulphate. When used these are present in an amount of ranging from 0 to about 50% by weight of the dosage form.

Flavors incorporated in the composition may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple, pear, peach, strawberry, raspberry, cherry, plum, pineapple, apricot and so forth. The amount of flavoring may depend on a number of factors, including the organoleptic effect desired. Flavors may be present in an amount ranging from about 0.05% to about 3% by weight based upon the weight of the dosage form.

Lubricants may also be used. Hydrophobic lubricants are preferred. Hydrophobic lubricants include, without limitation, calcium stearate, magnesium stearate, zinc stearate, stearic acid, stearowet C, mineral oil, vegetable oil, glyceryl behenate, sodium stearyl fumarate, talc, starch, and others. Hydrophilic lubricants include, without limitation, sodium benzoate, sodium chloride, sodium lauryl sulfate, magnesium lauryl sulfate, polyethylene glycol, and others. Magnesium stearate is preferred. These may be used in an amount of between about 0.5% and about 5% by weight, more preferably 0.5% to about 2.5% by weight of the dosage form. If desired the dosage form may also contain minor amounts of nontoxic substances such as wetting or emulsifying agents, pH buffering agents and the like, for example, sodium acetate, sorbitan monolaurate, triethanolamine, sodium acetate, triethanolamine oleate, sodium lauryl sulfate, dioctyl sodium sulfosuccinate, polyoxyethylene sorbitan fatty acid esters.

Glidants such as colloidal silicon dioxide may also be used to improve flow in conventional amounts of up to 5%, but preferably in an amount of about 1% or less.

Other active pharmaceutical ingredients (“OAPIs”) that may be used in accordance with the present invention in addition to lorazepam include, without limitation, analgesics, anti-inflammatories, antipyretics, antibiotics, antimicrobials, anxiolytics, laxatives, anorexics, antihistamines, antidepressants, antiasthmatics, antidiuretics, antiflatuents, antimigraine agents, antispasmodics, sedatives, antihyperactives, antihypertensives, tranquilizers, decongestants, beta blockers, peptides, proteins, oligonucleotides and other substances of biological origin, and combinations thereof. Also contemplated as OAPIs are the drugs and pharmaceutically active ingredients described in Mantelle, U.S. Pat. No. 5,234,957, in columns 18 through 21. That text of Mantelle is hereby incorporated by reference. The above-identified OAPIs may be coated onto the same carrier particle as the lorazepam or may be provided as a distinct particle. They may be coated or uncoated.

Tablets are preferred in accordance with one aspect of the present invention. In one embodiment, the tablets of the present invention have a hardness of about 15 Newtons or more, more preferably 20 Newtons or more, up to about 200 Newtons, more preferably 20 to about 100 Newtons, and a friability, as measured by the USP at the time of filing, of about 2% or less. In yet another embodiment, these tablets may include at least one nondirect compression carbohydrate as a filler. See U.S. Pat. No. 6,024,981. Preferably, these tablets are capable of rapidly disintegrating/dissolving in about 60 seconds or less, more preferably about 30 seconds or less as measured by the objective testing described herein, lorazepam containing particles can be swallowed as a dispersion, suspension or slurry. These tablets may be packaged in blister packages or in openable and reclosable multi-tablet packages.

The tablets of another embodiment of the invention often have a hardness of about 10 to about 20 Newtons, and a friability of more than 2% as measured by the U.S.P. method as of the filing date. Preferably these tablets are capable of rapidly disintegrating/dissolving in a patient's mouth in about 60 seconds or less, more preferably about 30 seconds or less as described above, such that the lorazepam containing particles can be swallowed as a dispersion, suspension or slurry.

Note that while the specification and claims may refer to a tablet of the invention as, for example, containing particles having a certain particle size or distribution, that recitation may be satisfied if the materials used prior to final blending and tablet formulation meet that recitation. In another example, while it might be difficult to know the weight gain of a coated lorazepam particle or its particle size distribution from an analysis of the finished dosage form, if it is determined that the protected lorazepam particles used to make the dosage form, prior to a final blending and compression steps, for example, did exhibit the desired coating level and/or particle size, that is sufficient.

Tablets can either be manufactured by direct compression, compression molding, wet granulation, dry granulation or any other tablet manufacturing technique. See, e.g., U.S. Pat. Nos. 5,178,878, 5,223,264 and 6,024,981 which are incorporated by reference herein.

### EXAMPLES

### Example 1

A lorazepam containing layering composition was prepared by mixing the following:

| | |
|---|---|
| water | 549.0 g |
| lorazepam | 21.3 g |
| hypromellose | 30.0 g |

Lorazepam was added to water while stirring. Hypromellose was slowly added while stirring and stirred for one hour. 600 g of sugar spheres (60/80) was placed in an MP1 fluid bed coater fitted with bottom spray and Wurster column. The layering material was pumped at a rate of 3 to 8 ml/min. Inlet air temperature was maintained at 55° C. Following application of the layering material, the layered product was dried in the fluid bed for 10 minutes.

To produce the over-coat solution:

| | |
|---|---|
| Water | 1140 g |
| Hypromellose | 60 g |

Hypromellose was slowly added to water while stirring and stirred for one hour to produce an over-coat solution. The over-coat solution was pumped at a rate of 3 to 5 ml/min into the same equipment. Following application of the over-coat solution, product was dried in the fluid bed for 10 minutes. Product was discharged into polyethylene bag and sealed.

The resulting protected lorazepam particles were used in the following formulations:

Flavor-free blend

| | QUANTITY | QUANTITY | |
|---|---|---|---|
| COMPONENT NAME | (wt %) | (g) | |
| Layer/coat Lorazepam (2.99% potent) | 16.72 | 50.2 | |
| Mannitol EZ, USP | 44.98 | 134.9 | |
| Mannitol, USP/EP/JP | 25.0 | 75.0 | |
| Crospovidone, NF/EP/JP | 6.0 | 18.0 | |
| Microcrystalline Cellulose, NF/EP/JP | 5.0 | 15.0 | |
| Sucralose, NF | 0.5 | 1.5 | |
| Silicon Dioxide, NF/EP/JP | 0.30 | 0.9 | |
| Magnesium Stearate, NF/EP/JP | 1.5 | 4.5 | |
| | 100.0% | 300.0 | g |

Mint-flavor blend

| | QUANTITY | QUANTITY | |
|---|---|---|---|
| COMPONENT NAME | (wt %) | (g) | |
| Layer/coat Lorazepam (2.99% potent) | 16.72 | 50.2 | |
| Mannitol EZ, USP | 44.48 | 133.4 | |
| Mannitol, USP/EP/JP | 25.0 | 75.0 | |
| Crospovidone, NF/EP/JP | 6.0 | 18.0 | |
| Microcrystalline Cellulose, NF/EP/JP | 5.0 | 15.0 | |
| Natural and Artificial Mint Flavor SN027513 | 0.5 | 1.5 | |
| Sucralose, NF | 0.5 | 1.5 | |
| Silicon Dioxide, NF/EP/JP | 0.30 | 0.9 | |
| Magnesium Stearate, NF/EP/JP | 1.5 | 4.5 | |
| | 100.0% | 300.0 | g |

In both cases, all ingredients except magnesium stearate were weighed, sieved through a 20-mesh screen, and mixed in an 1-qt V-blender for 30 minutes. The magnesium stearate was weighed, sieved through a 20-mesh screen, added to the V-blender and mixed for 5 minutes. Blends were discharged into polyethylene bags and sealed.

In both cases, flat-faced, bevel-edged, 5/16″ tablets were compressed on a rotary tablet press with target weight of 200 mg and target hardness of 30 N. Friability ˜0.7%.

### Example 2

A binder solution was made from the following:

| | |
|---|---|
| water | 450 g |
| povidone | 150 g |

Povidone was added to water while stirring and the mixture was stirred for 1 hour.

### Granulation A:

| | |
|---|---|
| lactose | 170.4 g |
| sodium bicarbonate | 4.0 g |
| lorazepam | 20.0 g |

Ingredients were weighed and placed in the bowl of a high-shear granulator. The bowl was sealed and the ingredients were dry mixed for one minute, impeller speed 300 rpm, chopper speed 3000 rpm. Maintaining the same speeds, 21.7 g of binder solution was added by peristaltic pump at a rate of 8 ml/min. Granulator was stopped and granulate was transferred to an MP1 fluid bed and dried (air inlet temperature 65° C.).

### Granulation B:

| | |
|---|---|
| microcrystalline cellulose | 151.0 g |
| polacrilin potassium | 4.0 g |
| lorazepam | 20.0 g |

Ingredients were weighed and placed in the bowl of a high-shear granulator. The bowl was sealed and the ingredients were dry mixed for one minute, impeller speed 300 rpm, chopper speed 3000 rpm. Maintaining the same speeds, 100 g of binder solution was added by peristaltic pump at a rate of 8 ml/min, followed by 20 g of water. Granulator was stopped and granulate was transferred to an MP1 fluid bed and dried (air inlet temperature 65° C.).

In both cases, the dried granulate was sieved through 35-mesh and 100-mesh screens and the −35/+100 fraction was retained for use.

### Weigh/blend:

Granulation A

| | QUANTITY | QUANTITY | |
|---|---|---|---|
| COMPONENT NAME | (wt %) | (g) | |
| Granulated Lorazepam (10.00% potent) | 10.00 | 30.0 | |
| Mannitol EZ, USP | 50.95 | 152.9 | |
| Mannitol, USP/EP/JP | 25.0 | 75.0 | |
| Crospovidone, NF/EP/JP | 6.0 | 18.0 | |
| Microcrystalline Cellulose, NF/EP/JP | 5.0 | 15.0 | |
| Natural and Artificial Orange Flavor | 0.75 | 2.2 | |
| SN027512 | | | |
| Sucralose, NF | 0.5 | 1.5 | |
| Silicon Dioxide, NF/EP/JP | 0.30 | 0.9 | |
| Magnesium Stearate, NF/EP/JP | 1.5 | 4.5 | |
| | 100.0% | 300.0 | g |

Granulation B

| | QUANTITY | QUANTITY | |
|---|---|---|---|
| COMPONENT NAME | (wt %) | (g) | |
| Granulated Lorazepam | 10.00 | 30.0 | |
| (10.00% potent) | | | |
| Mannitol EZ, USP | 50.95 | 152.9 | |
| Mannitol, USP/EP/JP | 25.0 | 75.0 | |
| Crospovidone, NF/EP/JP | 6.0 | 18.0 | |
| Microcrystalline Cellulose, NF/EP/JP | 5.0 | 15.0 | |
| Natural and Artificial Orange Flavor | 0.75 | 2.2 | |
| SN027512 | | | |
| Sucralose, NF | 0.5 | 1.5 | |
| Silicon Dioxide, NF/EP/JP | 0.30 | 0.9 | |
| Magnesium Stearate, NF/EP/JP | 1.5 | 4.5 | |
| | 100.0% | 300.0 | g |

In both cases, all ingredients except magnesium stearate were weighed, sieved through a 20-mesh screen, and mixed in an 1-qt V-blender for 30 minutes. The magnesium stearate was weighed, sieved through a 20-mesh screen, added to the V-blender and mixed for 5 minutes. Blends were discharged into polyethylene bags and sealed.

Tablet compression:

In both cases, flat-faced, bevel-edged, 5/16″ tablets were compressed on a rotary tablet press with target weight of 200 mg and target hardness of 30 N. Friability not measured.

### Example 3

The layer/coated tablets and granulated tablets as well as the active protected lorazepam particles were subjected to forced degradation. 5 tablets, in the case of tablets, and 100 to 270 mg in the case of active intermediates, were placed in open flasks at 80° C. in a convection oven for five days. Samples were assayed for potency at the end of five days and results were compared to non-stressed material from the same batch.

| | |
|---|---|
| Layer-coat active | −3.2% |
| Layer-coat, flavor-free tablet | −1.9% |
| Layer-coat, mint-flavor tablet | −2.9% |
| Granulate A | +1.6% |
| Granulate B | −0.0% |
| Granulate A tablet | −6.2% |
| Granulate B tablet | −5.3% |
| For comparison: | |
| Ativan (lorazepam), 1 mg | −9.5% |
| Mylan (lorazepam), 1 mg | −15.0% |

### Industrial Applicability

The invention is useful for preparing storage stable, orally disintegrable dosage forms (such as tablets) containing lorazepam as the active pharmaceutical ingredient. Furthermore, the invention permits the employment of granulation and/or layering using water that, despite the use of this solvent/carrier, the resulting tablets have excellent storage stability and disintegration times.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A storage stable, orally disintegrable dosage form comprising: protected lorazepam particles comprising lorazepam and a polymer having a glass transition temperature which is about 65° C. or more, said protected lorazepam particles being present in an amount sufficient to provide a therapeutically effective amount of lorazepam ranging from about 0.1 to about 100 mg per dosage form, at least one disintegrant selected from the group consisting of crosslinked PVP, a croscaramellose salt, a starch glycolate and an effervescent couple, and at least one carbohydrate based filler, said dosage form being capable of disintegrating within about 90 seconds or less as measured by U.S.P. and having a loss of potency of about 15% or less, as measured by forced degradation.

2. The dosage form of claim 1 wherein said protected lorazepam particle further comprises a carrier particle and wherein a mixture of said lorazepam and said a polymer having a glass transition temperature which is about 65° C. or more are disposed in a layer on said carrier particle.

3. The dosage form of claim 2 further comprising at least one coating disposed over said layer on said carrier particle.

4. The dosage form of claim 3 wherein said coating comprises a polymer having a glass transition temperature which is about 65° C. or more.

5. The dosage form of claim 4 wherein said coating and said layer comprise the same polymer having a glass transition temperature which is about 65° C. or more.

6. The dosage form of claim 1 wherein said protected particle is a granulate.

7. The dosage form of claim 6 wherein said granulate is a wet granulate.

8. The dosage form of claim 6 wherein said granulate further comprises a cogranulate and a binder.

9. The dosage form of claim 8 wherein said lorazepam is dissolved, suspended or dispersed in said binder.

10. The dosage form of claim 9 wherein said binder further comprises a polymer having a glass transition temperature which is about 65° C. or more.

11. The dosage form of claim 8 wherein said lorazepam is mixed with said cogranulate.

12. The dosage form of claim 11 wherein said binder comprises a polymer having a glass transition temperature which is about 65° C. or more.

13. The dosage form of claim 10 wherein said granulate further comprises a coating.

14. The dosage form of claim 13 wherein said coating comprises a polymer having a glass transition temperature which is about 65° C. or more.

15. The dosage form of claim 12 wherein said granulate further comprises a coating.

16. The dosage form of claim 15 wherein said coating comprises a polymer having a glass transition temperature which is about 65° C. or more.

17. The dosage form of claim 1 wherein said cellulose based material is HPMC.

18. The dosage form of claim 1 wherein said carbohydrate based filler is mannitol.

19. The dosage form of claim 18 wherein at least a portion of said mannitol is spray-dried mannitol.

20. The dosage form of claim 18 wherein said cogranulate material is selected from the group consisting of microcrystalline cellulose, carbonates, bicarbonates, carbohydrates and cellulosic materials and inert materials.

21. A storage stable, orally disintegrable tablet comprising: protected lorazepam particles comprising lorazepam and a cellulose based material or PVP, said protected lorazepam particles being present in an amount sufficient to provide a therapeutically effective amount of lorazepam ranging from about 0.1 to about 100 mg per tablet, at least one disintegrant selected from the group consisting of a crosslinked PVP, a croscaramellose salt, a starch glycolate and an effervescent couple, and at least one carbohydrate based filler, said tablet having a loss of potency of about 10.5% or less or measured by forced degradation and being either bioequivalent to nonorally disintegrable dosage form containing lorazepam at the same dose or being capable of disintegrating within about 60 seconds or less as measured by U.S.P.

22. The dosage form of claim 21 wherein said carbohydrate based filler is mannitol.

23. The dosage form of claim 21 wherein said protected lorazepam particles further comprise a solid support onto which said lorazepam and said cellulose based material or PVP are layered or a cogranulate with which said lorazepam is granulated using said cellulose based material or PVP as a binder.

24. The dosage form of claim 21 wherein said protected lorazepam particles comprise a cellulose based material selected from the group consisting of HPMC, HPC or EC.

25. A method of producing a storage stable lorazepam containing tablet comprising the steps of: layering a mixture of lorazepam and a GTT65 polymer onto a surface of a solid support to form a layered particle, said layer being provided in an amount sufficient to result in a percent weight gain of between about 5 and about 15%, coating said layered particle with a coating selected from a GTT65 polymer in an amount sufficient to result in a percent weight gain of between about 5 and about 15% so as to form protected lorazepam particles, blending said protected lorazepam particles in an amount sufficient to produce tablets coating between about 0.1 and about 100 mg of lorazepam per tablet, with at least one carbohydrate based filler and a disintegrated selected from a crosslinked PVP, a croscaramellose salt, a starch glycolate and a effervescent couple to form a blend, and compressing said blend into tablets containing about 0.1 and about 100 mg of lorazepam.

26. The method of claim 25 wherein said tablets are compressed to a hardness of between about 15 and about 200 Newtons, and a friability of about 2% or less.

27. The method of claim 25 wherein said tablets are compressed to a hardness of between about 10 and about 20 Newtons, and a friability of about 2% or more.

28. A method of producing a storage stable lorazepam containing tablet comprising the steps of wet granulating lorazepam and at least one cogranulate with a binder comprising a GTT65 polymer to form protected lorazepam particles; blending said protected lorazepam particles in an amount sufficient to produce tablets containing between about 0.1 and about 100 mg of lorazepam per tablet, with at least one carbohydrate based filler and a disintegrant selected from a crosslinked PVP, a croscaramellose salt, a starch glycolate and an effervescent couple to form a blend, and compressing said blend into tablets containing about 0.1 and about 100 mg of lorazepam.

29. The method of claim 28 wherein said tablets are compressed to a hardness of between about 15 and about 200 Newtons, and a friability of about 2% or less.

30. The method of claim 28 wherein said tablets are compressed to a hardness of between about 10 and about 20 Newtons, and a friability of about 2% or more.
